(19) United States
(12) Patent Application Publication
(10) Pub. No.: US 2024/0337580 A1
(43) Pub. Date: Oct. 10, 2024

(54) SYSTEM AND METHOD FOR ANALYZING FLOW CYTOMETRY RESULTS

(71) Applicant: Hoffmann-La Roche Inc., Little Falls, NJ (US)

(72) Inventors: Priscila CAMILLO TEIXEIRA, Basel (CH); José Manuel MARTINS ABRANTES TEIXEIRA DUARTE, Basel (CH); Raul RODRIGUEZ-ESTEBAN, Basel (CH); WeiQing Venus SO, Little Falls, NJ (US)

(73) Assignee: Hoffmann-La Roche Inc., Little Falls, NJ (US)

(21) Appl. No.: 18/291,790

(22) PCT Filed: Jul. 26, 2022
(86) PCT No.: PCT/EP2022/070888

§ 371 Date: Jan. 24, 2024

(30) Foreign Application Priority Data
Jul. 27, 2021 (EP) 21187934.1

(51) Int. Cl. G01N 15/1429; G16B 40/00; G16H 50/70

(52) U.S. Cl. CPC G01N 15/1429; G16B 40/00; G16H 50/70

(57) ABSTRACT

A system for determining a cell type and/or one or more functional markers of a cell using flow cytometry. A plurality of flow cytometry devices respectively perform flow cytometry of cells and use gating definitions at least in part different among each other, thereby generating gating definitions as respective results of the flow cytometry devices, which are at least partly inconsistent such that a same set of biomarkers detected by two different flow cytometry devices results in different gating definitions being output. A machine learning component receives the gating definitions as inputs, and generates a set of cell types and/or functional markers as an output and as a result of the flow cytometry analysis performed by the flow cytometry devices. The machine learning component has been trained using a set of manually curated training data comprising gating definitions resulting from the flow cytometry and corresponding cell types and/or functional markers.

### TECHNICAL FIELD

The present application generally relates to the field of flow cytometry, and more particularly to a system and a method for determining a cell type and/or one or more functional markers of a cell using flow cytometry.

### BACKGROUND

Flow cytometry is a technique used to detect and measure physical and chemical characteristics of a population of cells, in particular cell type and functional markers. A sample containing cells is suspended in a fluid and injected into the flow cytometer instrument. The sample is focused such that there is ideally flowing one cell at a time through a laser beam. The light, which is scattered or emitted by the cell, is characteristic to the cells and its components. Based on the scattered light the cell type and one or more of functional markers of the cell can be determined. With modern instruments tens of thousands of cells can be quickly examined and the data gathered are processed by a computer.

FIG. 1 schematically illustrates a flow cytometry apparatus 100. For a cell passing through the device its flow cytometric light scattering and fluorescence behavior are assessed. The forward scattered light is detected by a forward light scatter detector, and the side scattered light is detected by suitable detectors 1-4. The resulting data is processed automatically by a computer resulting in so called “gating definitions”, which represent the reportable results (or “reportables”) from a flow cytometry assay, as will be explained below.

Flow cytometry enables high content analysis of cell populations from heterogeneous samples through the identification of surface and intracellular antigen expression using fluorescent-labeled molecular probes and can provide insights in applications such as the identification of disease biomarkers, immune regulatory mechanisms and cellular signaling. Flow cytometry is an important tool in drug discovery and development in areas such as biomarker discovery, receptor occupancy and target engagement assays, and target-based and phenotypic screenings.

Recent years have seen tremendous development in multiplexing capabilities of flow cytometry instrumentation, in particular with the development of full spectrum flow cytometry. Using a polychromatic dispersion element to spread emitted light in front of the detector allows for full spectral analysis of a population of cells across a portion of the visible light spectrum. Different light dispersion and detection technologies have been used in attempts to increase the number of possible parameters that are identifiable in a system. Spectral flow cytometry systems are increasingly being implemented into biological workflows, and it meanwhile has reached the clinical space and is implemented in high parameter flow cytometry assays in multi-center clinical trials. Such global trials generate data from hundreds to thousands of samples across multiple flow cytometry assays that are capable of reporting on hundreds to thousands of different reportables as outputs.

Leveraging these new capabilities, for instance, pharmaceutical companies employ an evolving mix of flow cytometry assays during the drug project life cycle stemming from a range of internally-developed assays and potentially multiple external laboratories.

The outputs of the flow cytometry device (the “reportables”) typically are represented as a pattern of biomarkers and other descriptive elements, which are referred to as gating definitions. Based on the outputted gating definitions, which are automatically determined by the output data processing of the flow cytometry device or assay, there can be determined for each cell the cell type and the functional marker(s) of the cell flowing through the cytometry apparatus. The so-called “gating strategy”, which is applied in a certain assay, defines or specifies what markers are being used to identify cells of a certain type and functional markers.

The sharing of the biomarker data produced by these assays enables its reuse, reanalysis and reproducibility across different assays. Although much attention has been given to the harmonization and alignment of flow cytometry instruments in multi-center trials, there is still no guidance and/or tools for the standardization of flow cytometry data analysis and harmonization, such as the management of an assay's multiple reportable results (“reportables”). This increases the burden of deploying high parameter flow cytometry in the clinic, as well as data inconsistencies and errors that make cross-study analysis particularly difficult to execute.

Reportables, which are automatically outputted by flow cytometry assays, are typically represented by unstructured text strings, which are referred to as “gating definitions,” that comprise relevant markers and other information about the assay in a non-standardized format. Due to a lack of widespread standards, gating definitions can be written in multiple ways, which is an obstacle for data sharing and for using flow cytometry results from different makers.

Most individual gates in gating definitions name a marker that is detected, typically a protein. Marker names can be expressed using multiple synonyms. The so-called protein ontology (PRO) contains a comprehensive list of names and accurate synonyms, such as the gene name nomenclature, which identifies e.g. CD279 as synonym of “PDCD1”. Such ontologies can help in the standardization of marker names.

The Immunology Database and Analysis Portal (ImmPort) is a database, which receives data from the Human Immunology Project Consortium (HIPC), which is a multicenter collaboration aimed at performing large studies to profile human immune response to natural infection and vaccination. ImmPort has, among others, two fields, namely a) the cell population or cell type targeted, and b) the gating strategy being applied, which specifies which markers were being used to identify cells of that type.

Overton J A, Vita R, Dunn P, Burel J G, Bukhari S A C, Cheung K H, Kleinstein S H, Diehl A D. Peters B. Reporting and connecting cell type names and gating definitions through ontologies, BMC Bioinformatics. 2019 Apr. 25; 20 (Suppl 5): 182, recognized the problem of inconsistent gating definitions corresponding to cell types, and they proposed to use ontologies to cross-compare cell types and marker patterns. They used a large set of such gating definitions and corresponding cell types submitted by different investigators into ImmPort to examine the ability to parse gating definitions using terms from the Protein Ontology (PRO) and cell type descriptions, using the Cell Ontology (CL). Using their approach they identified clashes between populations. They further used logical axioms from CL to detect discrepancies between the two and proposed tentative standards on how to submit gating definitions and cell population names in the future that could be checked for validity and consistency in a fully automated fashion, thereby making cross examination of results from various assays easier and enabling the determination of cell types and functional markers based from flow cytometry assays in an automated fashion over multiple assays.

The approach of Overtone et al., however, has several problems. They introduced a harmonization approach for 4,388 gating definitions produced by a set of 28 academic centers. Their approach leveraged ontology mapping and, in particular, the Cell Ontology (CL) and the Protein Ontology (PRO). It involves, among other steps, mapping gating definitions to functional marker gene names and intensity levels using a rule-based method. As stated by the authors, however, pure rule-based approaches have shortcomings in dealing with mapping ambiguity. Due to incomplete ontologies, ontology mapping can lead to false negatives due to unmatched relevant concepts. Additionally, rule-based methods may struggle to capture complex relations between elements of the text.

There is therefore a need for a technology, which enables an automated determination of cell types and functional markers based on flow cytometry measurements of different assays with different, inconsistent gating definitions.

### SUMMARY

According to one embodiment there is provided a system for determining a cell type and/or one or more functional markers of a cell using flow cytometry, said system comprising:
a plurality of flow cytometry devices, which respectively perform flow cytometry of cells and which use gating definitions, which are at least in part different among each other, thereby generating gating definitions as respective results of the plurality of flow cytometry devices, which are at least partly inconsistent such that a same set of biomarkers detected by two different flow cytometry devices results in different gating definitions being outputted by the two different flow cytometry devices;
a machine learning component, which receives the gating definitions generated as results of the plurality of flow cytometry devices as inputs, and which generates, based on its training, a set of cell types and/or functional markers as an output of the machine learning component and thereby as a result of the flow cytometry analysis performed by the plurality of flow cytometry devices, wherein
the machine learning component has been trained using a set of training data, which has been manually curated, and which comprises gating definitions resulting from the flow cytometry performed by the flow cytometry devices and corresponding cell types and/or functional markers corresponding to the respective gating definitions.

Using this approach the problem of inconsistent gating definitions, which make the use of results from assays of different laboratories difficult, can be overcome. It enables the determination of cell types and functional markers through a large number of different assays, which may be located in different laboratories and which used different gating definitions.

According to one embodiment wherein the plurality of flow cytometry devices at least partly are located in different laboratories and/or are operated by different institutions or entities. Employing devices or assays, which are located in different laboratories or are operated by different entities, i. e. different research institutes or companies, enables integration of results from a wide range of experiments despite their inconsistent gating definitions.

According to one embodiment the machine learning component is implemented by choosing a ML pipeline with the aid of an automated ML (autoML) library, such as. E.g. the TPOTautoML library. This enables to choose a most efficient ML component for the given task.

According to one embodiment the training data set comprises a large number, at least several thousand, gating definitions about reportables from a plurality of assay panels from a plurality of different laboratories. The plurality of assay panels in one embodiment may comprise more than ten assays, in a further embodiment several dozens of assays, in an even further embodiment several hundred or several thousand assays. This enables the integration of wide range of different gating definitions.

According to one embodiment the training data set comprises a large number, at least several hundred, gating definitions about reportables from a plurality of assay panels from a plurality of different laboratories. This enables the integration of results from a wide range of experiments despite their inconsistent gating definitions

According to one embodiment the training data gating definitions have been manually annotated with corresponding cell types and functional markers. This ensures that the training data comprises a correct correspondence between gating definitions and cell types/functional markers.

According to one embodiment the annotated cell types are mapped to a consistent predefined cell type terminology, and/or to one or more multiple public ontologies. This increases the consistency of the annotation.

According to one embodiment gating definitions are pre-processed by one or more of i) transforming them to lowercase, ii) eliminating non-ASCII characters and the majority of non-alphanumeric characters. This enhances the performance of the ML component.

According to one embodiment a set of rules is applied to split tokenize gating definitions into units by identifying separator elements such that the tokens corresponded to individual gates.

According to one embodiment marker intensity definition including plus and minus signs next to individual gates, where they exist, are extracted for each token.

According to one embodiment the dataset then is divided into training and test sets, features for ML are based on all unique tokens produced by tokenization of the training dataset, and these features are then matched to all gating definitions in the training and testing sets to produce, respectively, the training and testing feature values.

According to one embodiment matches are not allowed when there are numerical boundaries around the match.

According to one embodiment marker intensity definitions are used to further refine feature values.

According to one embodiment there is provided a computer implemented method for determining a cell type and/or one of more functional markers of a cell using flow cytometry, said method comprising:
receiving data from a plurality of flow cytometry devices used in different laboratories, which respectively perform flow cytometry of cells and which use gating definitions, which are at least in part different among each other, thereby generating gating definitions as respective results of the plurality of flow cytometry devices, which are at least partly inconsistent such that a same set of biomarkers detected by two different flow cytometry devices results in different gating definitions being outputted by the two different flow cytometry devices;
using a machine learning component, which receives the gating definitions generated as results of the plurality of flow cytometry devices as inputs, and which generates, based on its training, a set of cell types and/or functional markers as an output of the machine learning component and thereby as a result of the flow cytometry analysis performed by the plurality of flow cytometry devices, wherein
the machine learning component has been trained using a set of training data, which has been manually curated, and which comprises gating definitions resulting from the flow cytometry performed by the flow cytometry devices and corresponding cell types and/or functional markers corresponding to the respective gating definitions.

### BRIEF DESCRIPTION OF THE DRAWINGS

The foregoing summary as well as the following detailed description of preferred embodiments are better understood when read in conjunction with the append drawings. For illustrating the invention, the drawings show exemplary details of systems, methods, and experimental data. The information shown in the drawings are exemplary and explanatory only and are not restrictive of the invention as claimed. In the drawings:

FIG. 1 shows a block diagram of a flow cytometry apparatus.

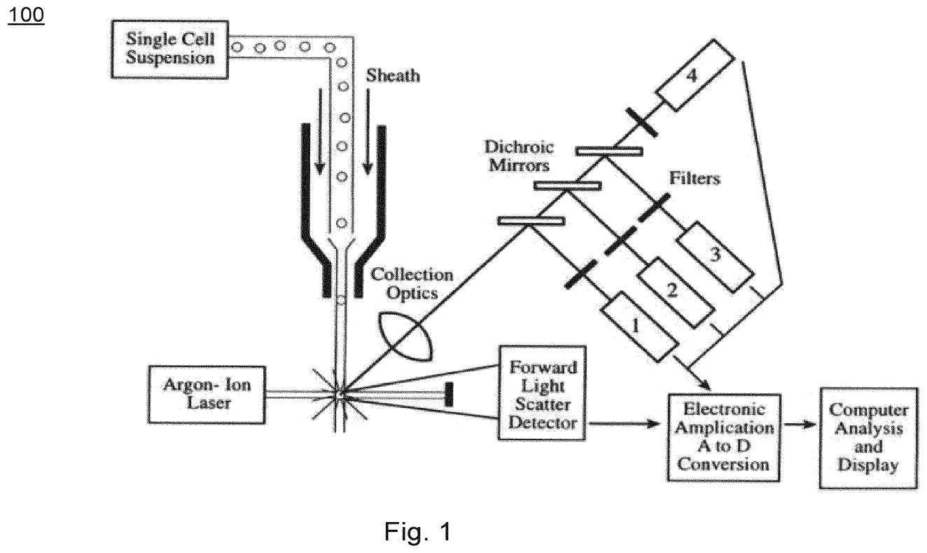

FIG. 2 schematically illustrates a system according to an embodiment.

FIG. 3 schematically illustrates the curation of training data and its use for prediction according to an embodiment.

FIG. 4 schematically illustrates histograms of AUROC values associated with the classification of each cell type and functional marker class.

### DETAILED DESCRIPTION

The present disclosure relates to systems and methods for a computer-implemented determination of cell types and functional markers based on flow cytometry results of multiple assays. For that purpose it enables the mapping of non-standardized gating definitions to standardized cell types and functional markers to thereby automatically determine the cell types and functional markers.

The cell types associated with each gating definition and the presence or absence of specific functional markers are of key interest for analyses concerning flow cytometry data. Gating definitions, as mentioned, are written in free text format and therefore require mapping to standard concepts to enable data integration and re-use over multiple assays.

In order to achieve this object, according to one embodiment there is applied a supervised machine learning (ML) approach to solve his problem. This approach is used for automatically identifying cell types and functional markers from gating definitions using an ML algorithm.

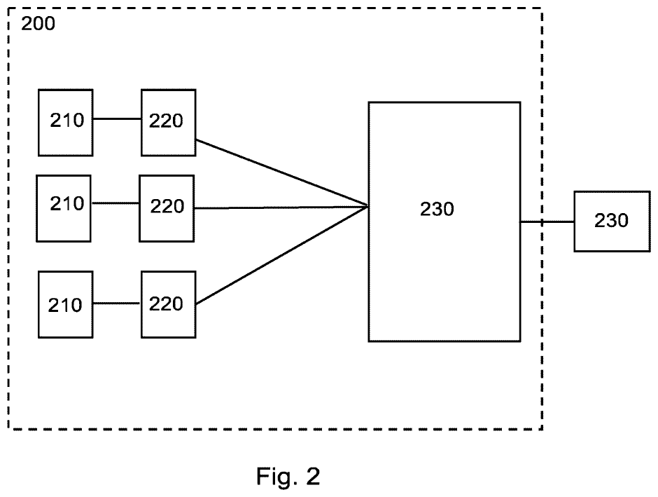

FIG. 2 shows a system 200 determining cell types and/or functional markers based on flow cytometry results, which are obtained by different assays. Several flow cytometry assays 210 produce as outputs respective gating definitions 220, which are non-standardized. They are inputted to a trained machine learning component 230, which has been trained by an appropriately prepared set of training data. Based on the training the component 230 is able to determine the cell types and functional markers, which correspond to the respective gating definitions 220, which have been generated as results of the various flow cytometry assays 210.

The plurality of flow cytometry devices 210 respectively perform flow cytometry of cells and use gating definitions, which are at least in part different among each other. They thereby generate gating definitions 220 as respective results of the plurality of flow cytometry devices 210, which are at least partly inconsistent such that a same set of biomarkers detected by two different flow cytometry devices results in different gating definitions being outputted.

A machine learning component 230 which receives the gating definitions generated as results of the plurality of flow cytometry devices as inputs. It generates, based on its training, a set of cell types and/or functional markers as an output of the machine learning component and hereby as a result of the flow cytometry analysis performed by the plurality of flow cytometry devices.

The machine learning component has been trained using a set of training data, which has been manually curated, and which comprises gating definitions resulting from the flow cytometry performed by the flow cytometry devices and corresponding cell types and/or functional markers corresponding to the respective gating definitions.

The machine learning component according to one embodiment is implemented by choosing a ML pipeline with the aid of an autoML (automated ML) library, e.g. the TPOT auto ML library. AutoML algorithms can help the end-to-end selection of an optimal pipeline of preprocessors, feature constructors, feature selectors, ML models and hyperparameter optimization for solving an ML task. The TPOT autoML library for classification selects a model from a list that, in its default configuration, includes Gaussian naïve Bayes, Bernoulli naïve Bayes, multinomial naïve Bayes, decision tree, extra trees, random forest, gradient boosting, K-nearest neighbors, linear support vector machine, logistic regression, extreme gradient boosting, stochastic gradient descent and multi-layer perceptron. The ML pipeline in one embodiment is selected by running the TPOT autoML algorithm on a training set. The selected pipeline then is primarily evaluated in the test set, which had not been seen by the autoML algorithm. In addition, in an embodiment it is evaluated by 10-fold cross-validation on the entire dataset.

In the following there will be described the preparation of the training data for the machine learning component according to one embodiment. In one embodiment, the training data set comprises several thousand, in a concrete example 4,849 gating definitions about reportables from several assays, in one concrete example 36 assay panels from a plurality of different laboratories. Despite assay differences, some gating definitions can be identical and, according to one embodiment, deduplication may be performed. In one concrete exemplary embodiment this resulted in a total of 3,045 unique gating definitions. Other numbers may be used in other exemplary embodiment, as will be recognized by the skilled person.

The unique gating definitions are then manually annotated by scientific experts with corresponding cell types and functional markers, in one concrete example embodiment this resulted in 117 unique cell types and 70 unique functional markers. Depending on the embodiment these annotations may initially lack some consistency, e.g. the same cell type or functional marker could be written in different ways by different experts. To increase consistency of annotation, according to one embodiment annotated cell types are mapped to a certain consistent predefined cell type terminology, which integrates domain experts' feedback and, according to one embodiment, to multiple public ontologies, which for example include one or more of Cell Ontology, BRENDA Tissue Ontology, SNOMED, NCI Thesaurus and MeSH.

According to one embodiment the mapping involves manual expert curation and, additionally, rule-based automated quality control. E.g., annotated marker gene names are harmonized to CD names where available. In a concrete exemplary embodiment this results, after the harmonization, in 56 unique cell types and 62 unique functional markers, which then are the target variables for the ML algorithm.

According to one embodiment generalizability and reproducibility is emphasized in building the overall prediction workflow. For that purpose, in one embodiment gating definitions are pre-processed by transforming them to lowercase, eliminating non-ASCII characters and most non-alphanumeric characters. For that purpose, according to one embodiment a set of rules is applied to split (“tokenize”) gating definitions into units by identifying “separator” elements. These units (“tokens”) often corresponded to individual gates (e.g. “CD3+CD4+CD25+” is split into the units CD3, CD4 and CD25). This process can be considered analogous to text tokenization, in which a text is split into lexical units called tokens. Marker intensity definitions (e.g. plus and minus signs next to individual gates, such as + in “CD3+”), where they exist, are extracted for each token.

The dataset then is divided into training (e.g. 80%) and test (e.g. 20%) sets. Features for ML are based on all unique tokens produced by tokenization of the training dataset. These features are then matched to all gating definitions in the training and testing sets to produce, respectively, the training and testing feature values.

In one embodiment matches are not Allowed when there are numerical boundaries around the match (e.g. the feature 45ra matches the gating definition “CD45RA+” but the feature cd4 does not). Marker intensity definitions are used to further refine feature values (e.g. a minus sign next to a matched token leads to a feature value of −1).

According to one embodiment ontology matching is applied on gating definitions to reduce feature-set cardinality. For this purpose, the Protein Ontology (PRO) release 62.0 in OWL format, which includes 331,920 terms, is downloaded from the Protein Ontology Consortium site (proconsortium.org).

According to one embodiment an ML pipeline is then chosen with the aid of the TPOT autoML (automated ML) library. Once the ML model has been chosen and trained, the resulting ML component can use new input data, results from various flow cytometry assays of various laboratories, and automatically can deliver as results of the flow cytometry analysis cell types and functional markers despite the inconsistent and non-standardized gating definitions form the various flow cytometry assays.

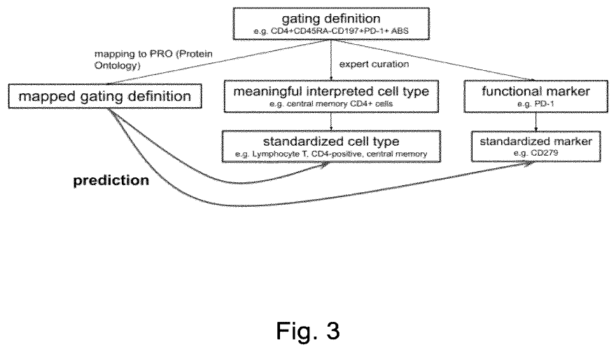

FIG. 3 schematically illustrates a process of curating training data and its use for prediction according to one embodiment of the invention. It shows the initial gating definitions resulting from various assays as the top box, which then are processed to generate the training data. This involves expert curation for identifying the cell types (“meaningful interpreted cell types”) and the functional markers, and it further in this embodiment involves the mapping of the gating definitions to some standardized format by mapping them to the protein ontology PRO. The expert curation may result in some inconsistencies resulting from different experts performing the curation, and therefore the “meaningful interpreted cell types” are further curated or homogenized to “standardized cell types” and the functional markers are further curated or homogenized to “standardized markers”. This results in the training set for the ML component, which can first be used for training and then, as shown in the figure, for prediction of results based on the mapped gating definitions.

In the following, the results of a concrete exemplary embodiment will be discussed. In this embodiment, a total of 3,043 gating definitions were manually annotated by scientific experts and harmonized to 56 unique cell types and 62 unique functional markers as shown in an excerpt of the concrete data in the following table 1.

TABLE 1
Examples of gating definitions mapped to cell types and markers.

| | | Functional |
|---|---|---|
| Gating definition | Cell type | marker |
| CD3 + CD4 + | Lymphocyte T, CD4- | CD25 |
| CD25_APC MFI | positive | |
| CD8 + PD-1 Tcell | Lymphocyte T, CD8- | CD279 |
| MESFNaHepLDTCL | positive | |
| Median CTLA4 in CD19 | Lymphocyte B | CD152 |
| %4-1BB in CD16 | Natural killer cell | CD137 |

Using this dataset, according to one concrete exemplary embodiment there was implemented a machine learning (ML)-based prediction workflow to predict the cell type and functional marker associated with a gating definition, i.e. solve a 56-class and a 62-class classification problem, respectively.

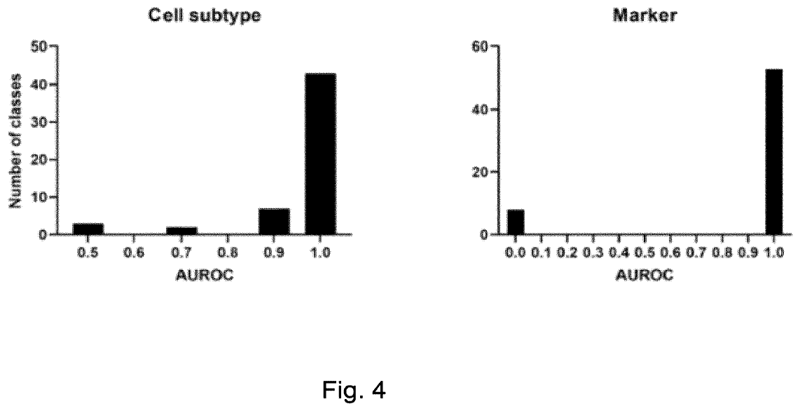

For cell type prediction, the data was split into training and testing datasets. Based on the gating definitions in the training dataset, a total of 281 features were created through data pre-processing steps such as tokenization described before. Feature values were extracted for both training and testing datasets to feed the ML pipeline. An ML pipeline was selected and optimized by the TPOT autoML (automated machine learning) algorithm. This pipeline was based on a stacking architecture composed of a random forest classifier and a logistic regression classifier. Using this pipeline, prediction accuracy on the test set was 97.2%. In the concrete exemplary implementation, the median AUROC (area under the curve of the receiver operating characteristic) for each class was 0.999 and the average AUROC was 0.95±0.12. Low performance for classes with few available gating definitions lowered the average AUROC. The histogram distribution of AUROC values for all the cell type classes is shown in FIG. 4, which shows the histogram of AUROC values associated with the classification of each cell type and functional marker class. The abscissa is labeled with the upper bound of the histogram intervals. The overall 10-fold cross-validation accuracy for the ML pipeline in this concrete implementation example was 94.2%.

Due to the way many manually-curated cell type annotations were written, in this embodiment these could be split into segments separated by commas. E.g., “Lymphocyte T, CD8-positive, regulatory” could be split into 3 segments: “Lymphocyte T,” “CD8-positive” and “regulatory” (see Table 1). An error analysis showed that, out of 177 classification errors made by the ML algorithm, 124 (70.1% of all errors) corresponded to discrepancies with the third segment of the class. E.g., predicting “Lymphocyte T, CD4-positive” when the actual class was “Lymphocyte T, CD4-positive, naive.”

In the concrete example, most predictions were correct with respect to the first segment, which corresponded to the broader cell type (e.g., monocyte, neutrophil, T-cell), except in 34 cases (19.2% of all errors).

Data availability by source (i.e. external laboratory or internal assay provider) varied widely, as can be seen in Table 2, which shows number and percentage of curated gating definitions available by source (i.e. external laboratory or internal assay provider).

TABLE 2

| Source | % | n |
|---|---|---|
| Internal | 3.0 | 92 |
| Laboratory 1 | 83.8 | 2551 |
| Laboratory 2 | 6.0 | 184 |
| Laboratory 3 | 7.1 | 216 |

To test the ability of an ML pipeline trained on data from one source to successfully make predictions on test data from another source (i.e. transfer learning), there have been performed several experiments. First, there was tested a pipeline on data from a single source after being trained on the rest of the sources. Results of this experiment in the first column in Table 3 indicate that lack of same-source data in the training set had a strong negative impact on pipeline performance.

Table 3 shows the accuracy of the prediction pipeline for cell types when tested on single-source data and trained on different sets of sources. The first column of results corresponds to a pipeline tested on 100% of the data available from a single source (same-source) and trained on data from the rest of the sources (other-source). The second column of results corresponds to an algorithm trained on 10% of same-source data plus 0% or 100% of other-source data. The third column of results corresponds to an algorithm trained on 50% same-source data plus 0% or 100% of other-source data

| Training data | % of existing | 0% | 10% | | 50% | |
|---|---|---|---|---|---|---|
| | same-source | | | | | |
| | % of existing | 100% | 0% | 100% | 0% | 100% |
| | other-sources | | | | | |
| Test data | Internal | 66.3 | 25.3 | 83.1 | 89.1 | 91.3 |
| | Laboratory 1 | 26.9 | 84.4 | 88.5 | 98.1 | 98.1 |
| | Laboratory 2 | 58.2 | 47.6 | 63.3 | 62.0 | 75.0 |
| | Laboratory 3 | 24.5 | 42.1 | 59.0 | 87.0 | 92.6 |

The second experiment involved mixing different amounts of same-source and other-source data in the training set. As can be seen in Table 3, the addition of more data to the training set, whether same-source or other-source, generally improved prediction accuracy. It also indicates that including even as little as 10% of same-source data significantly increased the prediction accuracy in most cases.

There was performed an analogous analysis with the prediction of functional marker annotations. For that purpose there were manually labeled 3,038 gating definitions with functional marker annotations and harmonized the marker names to 62 unique classes (see Table 1). This dataset was then used for creating, training and testing an ML pipeline as already described. An ML pipeline based on logistic regression with L2 regularization was selected by the TPOT autoML algorithm to map gating definitions to markers. The accuracy of the ML pipeline was 98.5% on the test set. The median AUROC was 1 and the AUROC average was 0.87±0.32. The overall 10-fold cross-validation accuracy was 95.0%.

Then there were carried the same experiments with marker prediction that were performed with cell type prediction in order to test the performance of the ML pipeline when trained on data from a mix of sources (see Table 4). Similarly to the case with cell types, an increase in training data availability, whether same-source or other-source, led to greater accuracy (with one exception).

| Training data | % of existing | 0% | 10% | | 50% | |
|---|---|---|---|---|---|---|
| | same-source | | | | | |
| | % of existing | 100% | 0% | 100% | 0% | 100% |
| | other-sources | | | | | |
| Test data | Internal | 26.1 | 38.6 | 74.7 | 91.3 | 97.8 |
| | Laboratory 1 | 15.5 | 83.1 | 83.8 | 98.8 | 96.8 |
| | Laboratory 2 | 81.5 | 70.5 | 86.7 | 94.6 | 98.9 |
| | Laboratory 3 | 81.9 | 74.9 | 97.4 | 92.6 | 98.1 |

Table 4 shows the accuracy of the prediction pipeline for markers when tested on single-source data and trained on different sources. The first column of results corresponds to an algorithm tested on 100% of the data available from a single source (same-source) and trained on data from the rest of the sources (other-source). The second column of results corresponds to an algorithm trained on 10% of same-source data plus 0% or 100% of other-source data. The third column of results corresponds to an algorithm trained on 50% same-source data plus 0% or 100% of other-source data.

Following Overton et al. (2019), in one embodiment there was also explored the potential use of a gene name ontology (Protein Ontology, PRO) to identify features that could derive from different synonyms from the same gene name. A total of 16 features corresponding to synonyms from 8 genes were merged (e.g. the features corresponding to ICOS and CD278 were merged into one feature). This, however, did not improve performance for either cell type or marker classification. From this one may conclude that the machine learning approach actually is superior to the ontology approach proposed by Overton et al. in terms of mitigating the effects of inconsistent gating definitions for various assays.

Concrete implementations have shown the feasibility and efficiency of using ML algorithms for mapping flow cytometry gating definitions to standardized cell types and functional markers, thereby enabling the integration of data provided by different assays deployed in multi-center studies that share flow cytometry data. More accurate and efficient data integration increases the value of data and enhances its ability to generate clinical and biological insights. Moreover, the ML proposed according to one embodiment can be re-trained as additional curated gating definitions are added to the training data to thereby further improving the analysis results.

While the training data that best helped mapping gating definitions was typically gating definitions from assays developed by the same laboratory, the experimental results show that the inclusion of gating definitions from assays from different laboratories in the training data generally provided additional prediction power, thereby showing the impact of transfer learning. Moreover, they show the significant achievement of enabling the automated detection of cell types and functional markers based on flow cytometry results, even if the flow cytometry assays are from different makers used in different laboratories, and employ different gating definitions.

Overall, in concrete example embodiments the mapping of functional markers showed better performance than the mapping of cell types, pointing towards differences in task complexity. Cell type mapping errors were most frequent in fine-grained cell subtypes as the main cell type was usually correctly predicted. As would be expected, mapping cell types or markers for which few examples were available in the training data was a challenge for the ML pipeline. This could be seen in the decreased AUROC for certain cell types with low number of samples. Additionally, the mapping of gating definitions from assays from laboratories for which there is no training data can lead to poorer performance due to differences in the way gating definitions are written. This can be addressed, in exemplary embodiments, by curating a small set of representative gating definitions, so that the algorithm can learn to recognize the feature patterns that define the gating definitions from the new laboratory. Thus, while generally “more data is better,” manually-curated data can, nonetheless, be gathered strategically to increase its representativeness and improve the performance of the ML pipeline at low cost.

According to on embodiment, the ML algorithm itself can help in identifying consistency errors in manual annotations if an error analysis is performed on its predictions. In one embodiment, the output of the ML algorithm is to be manually checked, which ensures high quality in the final output with minimal manual work. This output, in turn, according to one embodiment is used as additional, high quality training data.

A clear advantage of a purely ML approach over a rule-based approach is that it does not depend on the currency, comprehensiveness or quality of the rules or ontologies used in the latter. However, according to embodiments, the use of an ML approach does not preclude the inclusion of rules or ontologies. According to one embodiment, a mixed approach in which rules or ontologies are used to engineer features improve performance.

In the foregoing, there have been described embodiments, which use a machine learning ML component. Such an ML component according to one embodiment is implemented by an ML pipeline as explained in the following.

The process of machine learning is commonly arranged in a pipeline comprising the steps of data preprocessing, feature extraction and selection, and performing one or more machine learning algorithms. To deploy a complete machine learning pipeline, one or more machine learning models and their hyperparameters must be selected. Furthermore, parameters of the machine learning models may have to be adjusted through training the model. A deployment of a machine learning pipeline results in a machine learning component that can be used to perform a specific machine learning task.

In the following the creation of an ML pipeline using Auto ML will be described.

Selection of the machine learning pipeline, including specific machine learning models may be done in automated manner, for example using the AutoML method. AutoML selects for example one or more machine learning algorithms, their parameter settings, and the pre-processing methods suitable to detect complex patterns in input data of the machine learning task. One implementation of an AutoML method is the Tree-Based Pipeline Optimization Tool (TPOT) framework.

In the following examples the training methods will be described.

The machine learning models and/or the ensemble of machine learning models according to embodiments employ any suitable machine learning including one or more of: supervised learning (e.g., using logistic regression, using back propagation neural networks, using random forests, decision trees, etc.), unsupervised learning (e.g., using K-means clustering), semi-supervised learning, or reinforcement learning.

Example Machine learning techniques: Example machine learning techniques which can be used include the following.

### Clustering—Unsupervised Learning

In some examples, clustering methods can be used to cluster inputs. Clustering can be an unsupervised machine learning technique in which the algorithm can define the output. One example clustering method is K-means where K represents the number of clusters that the user can choose to create. Various techniques exist for choosing the value of K, such as for example, the elbow method.

### Dimensionality Reduction

Some other examples of techniques include dimensionality reduction. Dimensionality reduction can be used to remove the amount of information which is least impactful or statistically least significant. In networks, where a large amount of data is generated, and many types of data can be observed, dimensionality reduction can be used in conjunction with any of the techniques described herein. One example dimensionality reduction method is principle component analysis (PCA). PCA can be used to reduce the dimensions or number of variables of a “space” by finding new vectors which can maximize the linear variation of the data. PCA allows the amount of information lost to also be observed and for adjustments in the new vectors chosen to be made. Another example technique is t-Stochastic Neighbor Embedding (t-SNE).

### Neural Networks—Supervised Learning

Some other examples of techniques include the use of neural networks to perform a neural network task. A system receives training data corresponding to a neural network task. A neural network task is a machine learning task that can be performed by a neural network. The neural network can be configured to receive any type of data input to generate output for performing a neural network task. As examples, the output can be any kind of score, classification, or regression output based on the input. Correspondingly, the neural network task can be a scoring, classification, and/or regression task for predicting some output given some input.

The training data received can be in any form suitable for training a neural network, according to one of a variety of different learning techniques. Learning techniques for training a neural network can include supervised learning, unsupervised learning, and semi-supervised learning techniques. For example, the training data can include multiple training examples that can be received as input by a neural network. The training examples can be labeled with a known output corresponding to output intended to be generated by a neural network appropriately trained to perform a particular neural network task. For example, if the neural network task is a classification task, the training examples can be images labeled with one or more classes categorizing subjects depicted in the images.

To measure the accuracy of a candidate neural network, the system can use the training set to train the candidate neural network to perform a neural network task. The system can split the training data into a training set and a validation set, for example according to an 80/20 split. For example, the system can apply a supervised learning technique to calculate an error between output generated by the candidate neural network, with a ground-truth label of a training example processed by the network. The system can use any of a variety of loss or error functions appropriate for the type of the task the neural network is being trained for, such as cross-entropy loss for classification tasks, or mean square error for regression tasks. The gradient of the error with respect to the different weights of the candidate neural network can be calculated, for example using the backpropagation algorithm, and the weights for the neural network can be updated. The system can be configured to train the candidate neural network until stopping criteria are met, such as a number of iterations for training, a maximum period of time, convergence, or when a minimum accuracy threshold is met.

Aspects of this disclosure can be implemented in digital circuits, computer-readable storage media, as one or more computer programs, or a combination of one or more of the foregoing. The computer-readable storage media can be non-transitory, e.g., as one or more instructions executable by a cloud computing platform and stored on a tangible storage device.

In this specification the phrase “configured to” is used in different contexts related to computer systems, hardware, or part of a computer program. When a system is said to be configured to perform one or more operations, this means that the system has appropriate software, firmware, and/or hardware installed on the system that, when in operation, causes the system to perform the one or more operations. When some hardware is said to be configured to perform one or more operations, this means that the hardware includes one or more circuits that, when in operation, receive input and generate output according to the input and corresponding to the one or more operations. When a computer program is said to be configured to perform one or more operations, this means that the computer program includes one or more program instructions, that when executed by one or more computers, causes the one or more computers to perform the one or more operations.

Unless otherwise stated, the foregoing alternative examples are not mutually exclusive, but may be implemented in various combinations to achieve unique advantages. In the foregoing description, the provision of the examples described, as well as clauses phrased as “such as,” “including” and the like, should not be interpreted as limiting embodiments to the specific examples; rather, the examples are intended to illustrate only one of many possible embodiments.

Further embodiments are disclosed in the following examples.

Example 1: A system for determining a cell type and/or one or more functional markers of a cell using flow cytometry, said system comprising:
a plurality of flow cytometry devices, which respectively perform flow cytometry of cells and which use gating definitions, which are at least in part different among each other, thereby generating gating definitions as respective results of the plurality of flow cytometry devices, which are at least partly inconsistent such that a same set of biomarkers detected by two different flow cytometry devices results in different gating definitions being outputted by the two different flow cytometry devices;
a machine learning component, which receives the gating definitions generated as results of the plurality of flow cytometry devices as inputs, and which generates, based on its training, a set of cell types and/or functional markers as an output of the machine learning component and thereby as a result of the flow cytometry analysis performed by the plurality of flow cytometry devices, wherein
the machine learning component has been trained using a set of training data, which has been manually curated, and which comprises gating definitions resulting from the flow cytometry performed by the flow cytometry devices and corresponding cell types and/or functional markers corresponding to the respective gating definitions.

Example 2: The system of example 1, wherein the plurality of flow cytometry devices at least partly are located in different laboratories and/or are operated by different institutions or entities.

Example 3: The system of example 1 or 2, wherein the machine learning component is implemented by choosing a ML pipeline with the aid of an automated ML library.

Example 4: The system of any one of examples 1 to 3, wherein the training data set comprises a large number, at least several thousand, gating definitions about reportables from a plurality of assay panels from a plurality of different laboratories.

Example 5. The system of any one of examples 1 to 4, wherein for generating the training data gating definitions have been manually annotated with corresponding cell types and functional markers.

Example 6: The system of example 5, wherein to increase consistency of annotation, the annotated cell types are mapped to a consistent predefined cell type terminology, and/or to one or more multiple public ontologies.

Example 7: The system of any one of the preceding examples, wherein gating definitions are pre-processed by one or more of
i) transforming them to lowercase,
ii) eliminating non-ASCII characters and the majority of non-alphanumeric characters.

Example 8: The system any one of the preceding examples, wherein a set of rules is applied to tokenize gating definitions into units by identifying separator elements such that the tokens corresponded to individual gates.

Example 9: The system of example 8, wherein marker intensity definition including plus and minus signs next to individual gates, where they exist, are extracted for each token.

Example 10: The system of any one of the preceding examples, wherein the dataset then is divided into training and test sets, features for ML are based on all unique tokens produced by tokenization of the training dataset, and these features are then matched to all gating definitions in the training and testing sets to produce, respectively, the training and testing feature values.

Example 11. The system of example 10, where matches are not allowed when there are numerical boundaries around the match.

Example 12: The system of example 10 or 11, wherein marker intensity definitions are used to further refine feature values.

Example 13: A computer implemented method for determining a cell type and/or one of more functional markers of a cell using flow cytometry, said method comprising:
receiving data from a plurality of flow cytometry devices used in different laboratories, which respectively perform flow cytometry of cells and which use gating definitions, which are at least in part different among each other, thereby generating gating definitions as respective results of the plurality of flow cytometry devices, which are at least partly inconsistent such that a same set of biomarkers detected by two different flow cytometry devices results in different gating definitions being outputted by the two different flow cytometry devices;
using a machine learning component, which receives the gating definitions generated as results of the plurality of flow cytometry devices as inputs, and which generates, based on its training, a set of cell types and/or functional markers as an output of the machine learning component and thereby as a result of the flow cytometry analysis performed by the plurality of flow cytometry devices, wherein
the machine learning component has been trained using a set of training data, which has been manually curated, and which comprises gating definitions resulting from the flow cytometry performed by the flow cytometry devices and corresponding cell types and/or functional markers corresponding to the respective gating definitions.

Example 14: The computer implemented method of example 1, further comprising the features as additionally defined in one of examples 2 to 12.

1. A system for determining a cell type and/or one or more functional markers of a cell using flow cytometry, said system comprising:
a plurality of flow cytometry devices, which respectively perform flow cytometry of cells and which use gating definitions, which are at least in part different among each other, thereby generating gating definitions as respective results of the plurality of flow cytometry devices, which are at least partly inconsistent such that a same set of biomarkers detected by two different flow cytometry devices results in different gating definitions being outputted by the two different flow cytometry devices;
a machine learning component, which receives the gating definitions generated as results of the plurality of flow cytometry devices as inputs, and which generates, based on its training, a set of cell types and/or functional markers as an output of the machine learning component and thereby as a result of the flow cytometry analysis performed by the plurality of flow cytometry devices, wherein
the machine learning component has been trained using a set of training data, which has been manually curated, and which comprises gating definitions resulting from the flow cytometry performed by the flow cytometry devices and corresponding cell types and/or functional markers corresponding to the respective gating definitions.

2. The system of claim 1, wherein the plurality of flow cytometry devices at least partly are located in different laboratories and/or are operated by different institutions or entities.

3. The system of claim 1, wherein the machine learning component is implemented by choosing a ML pipeline with the aid of an automated ML library.

4. The system of claim 1, wherein the set of training data comprises a large number, at least several thousand, gating definitions about reportables from a plurality of assay panels from a plurality of different laboratories.

5. The system of claim 1, wherein for generating the training data, gating definitions have been manually annotated with corresponding cell types and functional markers.

6. The system of claim 5, wherein to increase consistency of annotation, the annotated cell types are mapped to a consistent predefined cell type terminology, and/or to one or more multiple public ontologies.

7. The system of claim 1, wherein gating definitions are pre-processed by one or more of
i) transforming the gating definitions to lowercase,
ii) eliminating non-ASCII characters and the majority of non-alphanumeric characters.

8. The system of claim 1, wherein a set of rules is applied to tokenize gating definitions into units by identifying separator elements such that the tokens correspond to individual gates.

9. The system of claim 8, wherein a marker intensity definition including plus and minus signs next to individual gates, where they exist, is extracted for each token.

10. The system of claim 1, wherein the set of training data is divided into a training dataset and a test dataset, features for machine learning are based on all unique tokens produced by tokenization of the training dataset, and the features are then matched to all gating definitions in the training dataset and the testing dataset to produce, respectively, training feature values and testing feature values.

11. The system of claim 10, wherein matches are not allowed when there are numerical boundaries around the match.

12. The system of claim 10, wherein marker intensity definitions are used to further refine feature values.

13. A computer implemented method for determining a cell type and/or one of more functional markers of a cell using flow cytometry, said method comprising:
receiving data from a plurality of flow cytometry devices used in different laboratories, which respectively perform flow cytometry of cells and which use gating definitions, which are at least in part different among each other, thereby generating gating definitions as respective results of the plurality of flow cytometry devices, which are at least partly inconsistent such that a same set of biomarkers detected by two different flow cytometry devices results in different gating definitions being outputted by the two different flow cytometry devices;
using a machine learning component, which receives the gating definitions generated as results of the plurality of flow cytometry devices as inputs, and which generates, based on its training, a set of cell types and/or functional markers as an output of the machine learning component and thereby as a result of the flow cytometry analysis performed by the plurality of flow cytometry devices, wherein
the machine learning component has been trained using a set of training data, which has been manually curated, and which comprises gating definitions resulting from the flow cytometry performed by the flow cytometry devices and corresponding cell types and/or functional markers corresponding to the respective gating definitions.
